(19) United States
(12) Patent Application Publication
(10) Pub. No.: US 2024/0009209 A1
(43) Pub. Date: Jan. 11, 2024

(54) PROTEIN-BOUND CANNABINOID FORMULATIONS AND USES THEREOF

(71) Applicant: YISSUM RESEARCH DEVELOPMENT COMPANY OF THE HEBREW UNIVERSITY OF JERUSALEM LTD, Jerusalem (IL)

(72) Inventors: Yechezkel BARENHOLZ, Jerusalem (IL); Ahuva CERN, Modiin (IL)

(21) Appl. No.: 18/247,494

(22) PCT Filed: Sep. 30, 2021
(86) PCT No.: PCT/IL2021/051179

§ 371 Date: Mar. 31, 2023

Related U.S. Application Data
(60) Provisional application No. 63/086,223, filed on Oct. 1, 2020.

(51) Int. Cl. A61K 31/00; A61K 47/42; A61K 9/00

(52) U.S. Cl. CPC A61K 31/658; A61K 47/42; A61K 9/0019

(57) ABSTRACT

The present disclosure provides formulations comprising a holding medium and homogeneously dispersed therein protein-bound cannabinoid, wherein the cannabinoid and the protein in the protein-bound cannabinoid are at a weight ratio of at least 10 mg cannabinoid to 50 mg protein (10:50 w/w). The formulations have shown to be in the form of particles enabling upon local injection prolonged release of the cannabinoid. Also provided by the present disclosure is a method of obtaining the protein-bound cannabinoid and uses thereof.

### TECHNOLOGICAL FIELD

The present disclosure concerns formulations of cannabinoids.

### BACKGROUND ART

References considered to be relevant as background to the presently disclosed subject matter are listed below:

International patent application publication No. WO2015/140736

International patent application publication No. WO2018/167795

Chinese patent application publication No. CN110302179

Chinese patent application publication No. CN110664622

International patent application publication No. WO2020/167751

Acknowledgement of the above references herein is not to be inferred as meaning that these are in any way relevant to the patentability of the presently disclosed subject matter.

### BACKGROUND

Delta-9-Tetrahydrocannabinol (THC), Cannabidiol (CBD), Cannabinol (CBN), Cannabichromene (CBC), Cannabigerol (CBG) and about 80 other chemicals are all in a class of compounds known as cannabinoids, found in abundance in the cannabis plant. Cannabinoids are responsible for many of the effects of cannabis consumption and have important therapeutic benefits. For example, CBD is known to provide relieve for chronic pain, thus, offering relief to patients with multiple sclerosis, fibromyalgia, and epilepsy, as well as to anxiety related disorders.

Most cannabinoids are lipid soluble and are available for oral delivery. Attempts have been made to improve solubility of CBD.

WO2015/140736 describes protein-bound cannabinoid and methods for their preparation. Specifically, cannabinoid solution or cannabis extract is combined with an aqueous solution or suspension comprising a plasma protein, such as albumin, to form a protein-bound cannabinoid.

WO2018/167795 describes the formation of compositions comprising cannabinoids bound to plasma proteins, such as albumin. The composition was administered orally or by intraperitoneal injection.

CN110302179 describes oil in water nano-emulsions of CBD with albumin, made for the purpose of improving water solubility and biocompatibility of CBD.

CN110664622 describes a moisturizing spray containing a combination of CBD with albumin so as to form a water-soluble for CBD. The albumin-CBD are in a form of oil in water nano-emulsions.

WO2020/167751 describes cannabinoid compositions comprising cannabinoid bound to a peptide through a linker to form a cannabinoid peptide complex. The composition is described for oral administration.

### GENERAL DESCRIPTION

The present disclosure provides, in accordance with a first of its aspects, a formulation comprising a holding medium and homogeneously dispersed therein protein-bound cannabinoid, wherein said cannabinoid and said protein in the protein-bound cannabinoid are at a weight ratio of at least 10:50 (i.e. 10 mg cannabinoid for every 50 mg of protein).

Further provided by the present disclosure, is a method of obtaining protein-bound cannabinoid entities, the method comprises mixing an aqueous medium of said protein with at least cannabinoid compound to form a mixture, wherein said mixing comprises at least one vigorous mixing stage the mixture comprising a cannabinoid to protein weight ratio of at least 10:50 (i.e. at least 10 mg cannabinoid to 50 mg protein).

In accordance with a third aspect, the present disclosure provides a method of treatment, the method comprising administering to a subject in need of said treatment a formulation as disclosed herein.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1E are microscope images (microscope Zeiss SN 221209×200 magnification)of CBD-HSA (50 mgCBD/50 mgHSA) over different stirring times, including 1 hour of stirring @ 25° C.) (FIG. 1A), 24 hours of stirring @ 4° C. (FIG. 1B), and 48 hours stirring @ 4° C. (FIG. 1C). as well as CBD-HSA (100 mgCBD/50 mgHSA) after stirring at 4° C., for 48 hours (FIG. 1D), and further CBD-HSA (250 mgCBD/50 mgHSA) stirred for 10 days at 4° C. (FIG. 1E).

FIGS. 2A-2F. are microscope images (microscope Zeiss SN 221209×200 magnification) of CBD-HSA (50 mgCBD/50 mgHSA) following homogenization, taken after different durations, including 5 minutes (FIG. 2A), 10 minutes (FIG. 2B) 15 minutes (FIG. 2C) 20 minutes (FIG. 2D) 30 minutes (FIG. 2E) and 60 minutes (FIG. 2F).

FIGS. 3A-3F are microscope images (microscope Zeiss SN 221209×200 magnification) of CBD-HSA (50 mgCBD/50 mgHSA) following sonication, taken after different durations, including 1 minutes (FIG. 3A), 5 minutes (FIG. 3B) 10 minutes (FIG. 3C) 15 minutes (FIG. 3D) 25 minutes (FIG. 3E) and 30 minutes (FIG. 3F).

FIGS. 4A-4H are microscope images (microscope Zeiss SN 221209×200 magnification) of CBD-HSA (100 mgCBD/50 mgHSA, 200 mgCBD/50 mgHSA, 300 mgCBD/50 mgHSA and 400 mgCBD/50 mgHSA) after 1 minute sonication (FIGS. 4A, 4B, 4C and 4D respectively), or following sonication for 1 minute and thereafter 48 hours of stirring at 4° C. (FIGS. 4E, 4F, 4G and 4H, respectively).

FIGS. 5A-5B are light microscope images (×200 magnification) before and after lyophilization of CBD -HSA (167mgCBD/50mgHSA), respectively.

FIG. 6 is a CryoTEM image of CBD-HSA particles after size reduction treatment

FIG. 7 is a light microscope image (×200 magnification) of CBD-mouse albumin (CBD-MA) (50 mgCBD/50 mgMA) after 5 days of stirring.

FIG. 8A-8C are microscope images (×200 magnification) of CBD-IVIg (50 mgCBD/50 mgHSA) after 24 h of stirring (4° C., FIG. 8A) and 48 hours stirring (4° C., FIG. 8B), as well as CBD-IVIg (100 mgCBD/50 mgHSA) after stirring at 4° C., for 48 hours (FIG. 8C).

FIG. 9 is a graph showing mice plasma CBD concentrations (ng/ml) after IV administration of 12 mg/kg dose vs the different IM formulations (n=3, mean±SD, w/o outlier)

FIGS. 10A-10B are graphs showing absolute CBD released (mg) from the muscles after IM administration of the different formulations (n=3, mean±SD, w/o outlier) (FIG. 10A) and Percent CBD released from the muscles after IM administration of the different formulations (n=3, mean±SD, w/o outlier) (FIG. 10B).

FIG. 11 is a graph showing mice plasma profile after IM administration of CBD-HSA or SC administration of CBD-MA.

FIG. 12 is a graph showing plasma CBD concentrations following SC administration of 5 mg/kg CBD-CA dose to a dog.

### DETAILED DESCRIPTION

Cannabinoids in general, and specifically CBD, are lipophilic compounds and as such the formulation development strategy usually relies on its solubility in lipids. For example, Epidiolex (CBD oral solution) is based on sesame oil. Marinol (Dronabinol), FDA approved cannabinoid for oral administration, is formulated as capsules containing a solution in sesame oil.

For the development of injectable formulation, the continuous phase of the formulation should be preferably aqueous, and as such present a challenge for cannabinoids injectable formulation development.

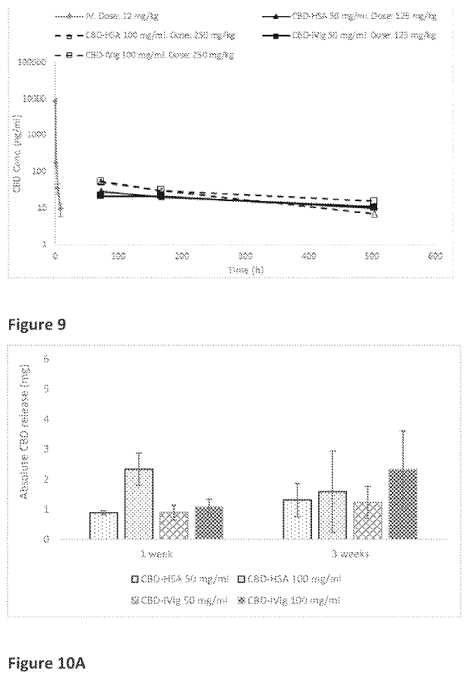

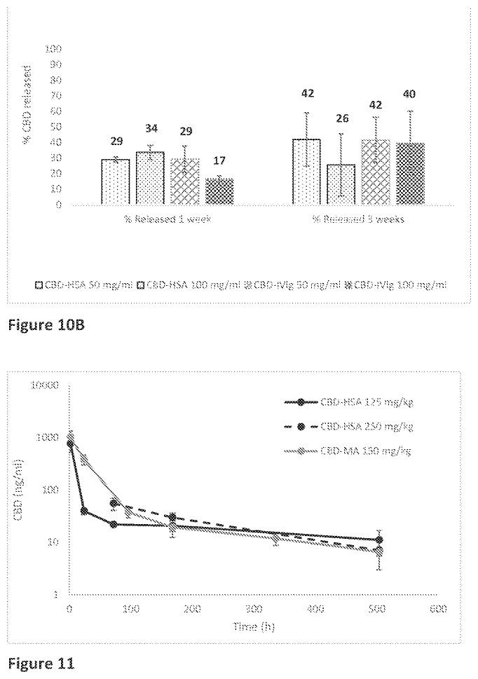

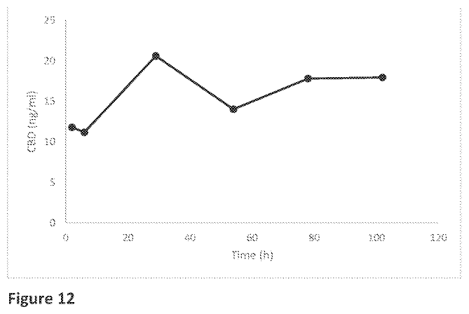

The present disclosed the ability to disperse high CBD concentrations in iso-osmotic solutions (5%) of a serum protein, including albumins including and not limited to mouse albumin (MA), Dog albumin (Canine, CA) and human serum albumin (HSA) and immunoglobulins (IVIg) in a manner allowing the formation of a homogeneous dispersion with a narrow size distribution that allows the injection of the formulation, e.g. by intramuscular (IM) or subcutaneous (SC) or intradermal (ID) routes, with a demonstrated prolonged plasma levels of CBD in mice for at least 3 weeks (See for example, FIGS. 9 and 11) and in dog, for at least 4 days (See, for example, FIG. 12).

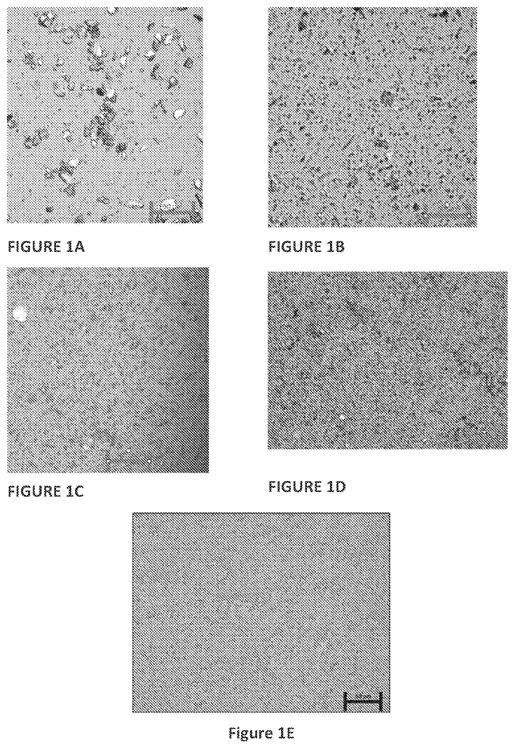

Further, the high concentration of the cannabinoid with respect to the protein carrier (see for example, FIG. 1E) was unexpected and surprising as usually the amount of carrier is much greater than that of the carried payload.

Thus, in accordance with a first of its aspects, the present disclosure provides a formulation comprising a holding medium and homogeneously dispersed therein protein-bound cannabinoid, wherein said cannabinoid and said protein in the protein-bound cannabinoid are at a weight ratio of at least 10:50 (i.e. at least 10 mg cannabinoid for every 50 mg protein, and preferably higher, e.g. 30 mg cannabinoids for every 50 mg protein, or 40 mg cannabinoids for every 50 mg protein or even more than 50 mg cannabinoid, as further discussed below).

The holding medium can be any aqueous based medium suitable for holding therein the protein bound cannabinoid. In some examples, the medium is one suitable for storage of the suspension of protein-bound cannabinoid. In some other examples, the medium is one suitable for administration of the protein-bound cannabinoids, e.g. a physiologically acceptable carrier.

In some examples, the holding medium is Alburex® (Alburex® 5 and Alburex® 25, Albumin (Human, dogs, horses and mice and also from other domestic animals), are sterile aqueous solutions of albumin obtained from large pools of adult human dogs, mice etc., venous plasma by low temperature-controlled fractionation according to the Cohn process modified by Kistler Nitschmann. It is stabilized with sodium acetyltryptophanate and sodium caprylate and pasteurized at 60° C. for at least 10 hours).

In the context of the present disclosure a protein is any amino acid containing molecule. The protein can be a short protein, i.e. an oligopeptide containing even only few amino acid units. Further, in the context of the present disclosure, the protein is a priori, a water-soluble protein with a solubility that allows the formation of an iso-osmotic solution. For example, the protein may be albumin, e.g. human serum albumin

The protein can be a naturally occurring protein, a fragment of a naturally occurring protein, a modified naturally occurring protein, or a functional homologue of a naturally occurring protein. The protein can also be a synthetic protein. Thus, in the context of the present disclosure, when referring to a protein, it is to be understood as encompassing a naturally occurring protein as well as any one of the alternatives outlined below.

The protein can be selected based on its capability to form, with the selected cannabinoid a dispersion. In other words, its ability to homogenously dispersed the cannabinoids added to an aqueous medium of the protein.

In some examples, the protein is a serum protein.

In some examples, the protein is of human source.

In some examples, the protein is of any animal, yet non-human source, e.g. bovine, canine, porcine, horse, or from other animals, such as domestic animals, etc.

In yet some other examples, the protein is a plant protein.

In some preferred examples, the protein is a serum protein. In one example, the serum protein is albumin; in another example, the serum protein includes globulins. When the protein is a globulin, this includes any one of alpha, beta, and gamma globulins.

The protein in bound to the cannabinoid. It is to be understood that in the context of the present disclosure when referring to the binding of the protein to the cannabinoid, it is to be understood as encompassing any form of connection/association that is not include covalent binding of the two. In other words, the binding is a non-covalent binding.

In some examples, the protein and the cannabinoid form a non-covalent complex. When referring to a complex it is to be understood to mean that cannabinoid and the protein are physically, yet not covalently, associated. Without being bound thereto the complex (i.e. the particulate form containing the complex) may include a plurality of protein molecules and a plurality of cannabinoid compounds. A unique finding of the present disclosure is the fact that the cannabinoid and the protein complex and form into a particle. Without being bound thereto, it is assumed that the formation into particulate form allows for the prolonged delivery of the cannabinoid.

Cannabinoids are well known in the art. In the context of the present disclosure, when referring to cannabinoid, it is to be understood as encompassing a single compound or a combination of cannabinoid compounds (i.e. the term as used herein encompasses a single or a plurality of such compounds). In some examples, the combination of cannabinoids comprises components of the plant extract, i.e. multiple cannabinoids and optionally plant flavonoids and terpenoids.

In some examples, the cannabinoid is or comprises cannabidiol (CBD).

In some other examples, the cannabinoid is or comprises tetrahydrocannabinol (THC) (Delta9-THC and/or Delta8-THC).

Other cannabinoids that fall within the scope of the present disclosure include one or any combination of two or more cannabinoids selected from the group consisting of cannabigerol (CBG), cannabigerolic acid (CB GA), cannabigerol monomethyl ether (CB GM), cannabichromene (CBC), cannabichromanone (CBCN), cannabichromenic acid (CBCA), cannabivarichromene (CBCV), cannabichromevarinic acid (CBCVA), isotetrahydrocannabinol (iso-THC), cannabinol (CBN), cannabinolic acid (CBNA), cannabinol methyl ether (CBNM), cannabinol C₄ (CBN-C₄), cannabinol C₂ (CBN-C₂), cannabinol C₁ (CBN-C₁), cannabinodiol (CBND), cannabielsoin (CBE), cannabielsoic acid A (CBEA-A), Cannabielsoic acid B (CBEA-B), cannabicyclol (CBL), cannabicycloic acid (CBLA), cannabicyclovarin (CBLV), cannabitriol (CBT), cannabitriolvarin (CBTV), ethoxy-cannabitriolvarin (CBTVE), cannabivarin (CBV), cannabinodivarin (CBVD), tetrahydrocannabivarin (THCV), cannabidivarin (CBDV), cannabigerovarin (CBGV), cannabigerovarinic acid (CBGVA), cannabifuran (CBF), dehydrocannabifuran (DCBF), cannabirispol (CBR), each constituting a separate embodiment of the present disclosure.

In some examples, the cannabinoid is or comprises a combination of CBD and any one or more of the above listed cannabinoids.

In some preferred examples, the cannabinoid within the formulation is CBD.

The term CBD compound encompasses, in the context of the present disclosure, CBD as well as functional homologues thereof. When referring to a CBD functional homologue it is to be understood as a compound having similar physico-chemical properties as CBD.

In some examples, the CBD functional homologue is a chemical analogue of CBD containing at least one benzene ring and a logP above 4.

In some examples, a CBD functional homologue includes structural homologue (including isomers) of CBD that, similar to CBD is lacking the psycho-activity of Tetrahydrocannabinol (THC).

In some examples, the CBD compound is a natural phytocannabinoids.

In some examples, the CBD compound is a synthetic CBD homologue.

Non-limiting examples of CBD compounds include name 2-[(1R,6R)-6-Isopropenyl-3-methylcyclohex-2-en-1-yl]-5-pentylbenzene-1,3-diol (CBD), the synthetic Cannabidiol-dimethylheptyl (CBD-DNH), the phytocannabinoids Cannabidivarin (CBDV), Cannabidivarinolic acid (CBDVA), Cannabidiol monomethyl ether (CBDM) [Paula Morales, Patricia H. Reggio, and Nadine Jagerovic “An Overview on Medicinal Chemistry of Synthetic and Natural Derivatives of Cannabidiol” Front Pharmacol.” 8:422, (2017)].

In some examples, the active ingredient is CBD known by its chemical name 2-[(1R,6R)-6-Isopropenyl-3-methylcyclohex-2-en1-yl]-5-pentylbenzene-1,3-diol.

The formulation comprising protein bound cannabinoids can comprise different pairs or protein-cannabinoids. Thus, a single formulation can comprise either only a specific type of protein, e.g. only human serum albumin (HSA); and specific type of cannabinoid, e.g. only CBD, in the associated protein-cannabinoid entity, or it can comprise different proteins bound to a specific cannabinoid, e.g. HSA and immunoglobulin both bound to CBD, or a single protein bound to different cannabinoids, e.g. HSA bound to different cannabinoids, or a combination of different pairs of protein and cannabinoids.

As noted above, the formulation is defined by its cannabinoid to protein weight ratio, being at least 10:50. In the context of the present disclosure, when referring to a weight ratio of 10:50 (e.g. 10 mgCBD: 50 mgHSA) it is to be understood that there as at minimum 10 mg cannabinoid for every 50 mg protein, and yet, preferably more than 10 mg cannabinoid for every 50 mg protein.

In some examples, the weight ratio is at least 20:50; at times the weight ratio is at least 30:50 at times; at times the weight ratio is at least 40:50; at times, even at least 50:50; at times at least 60:50; at times at least 70:50; at times at least 80:50; at times at least at times at least 90:50; at times at least 100:50; at times at least 110:50; at times at least 120:50; at times at least 130:50; at times at least 140:50; at times at least 150:50; at times at least 160:50; at times at least 170:50; at times at least 180:50; at times at least 190:50; at times at least 200:50; at times, at least 250:50; at times at least 300:50; at times, at least 350:50 at least 400:50; or even at least 400:50.

In some examples, the weight ratio is within a range of 10 mg:50 mg to 500 mg:50 mg; at times, within a range of 20:50 and 500:50; at times within a range of and 500:50 or any range within this range; at times, within a range of 30:50 and 250:50; at times within a range of 30:50 and 400:50; at times within a range of 50:50 and 400:50; at times within a range of 100:50 and 500:50.

The cannabinoid to protein mole ratio can also be calculated. For example, for CBD:HSA the calculation will be: HSA molecular weight is 66.5 KDa and that of CBD is 314.47 g/mol, so one can calculate CBD to HSA mole ratio to be carry ˜206 (HSA 50 mg CBD 50 mg 159 mmole, the mole ratio is 159/0.77=206) which means that a molecule of HSA carries 206 molecules of CBD.

The protein bound cannabinoid within the formulation is in a form of particles within a dispersion. The particles comprising the protein-bound cannabinoid are characterized by their narrow range of size distribution.

In some examples, the particles are defined by their mean size (diameter), being up to 10 μm.

In some examples, the particles are defined by a mean diameter of at least 0.1 μm. In some examples, the particles are defined by a mean diameter of at least 0.2 μm. In some examples, the particles are defined by a mean diameter of at least 0.3 μm. In some examples, the particles are defined by a mean diameter between about 0.1 μm and about 0.5 μm. Such size range would be particular suitable for IV injection.

In some examples, the particles are defined by their d50 value (i.e. the dimensions of at least 50% of the particles) being within a range of 0.1 μm and 0.3 μm.

In some examples, the particles are defined by a mean diameter within a range of 1 μm and 10 μm. In some examples, the mean particles size is in the range 1 μm and 7 μm; at times within a range of 2 μm and 7 μm; at times within a range of 1 μm and 5μm; at times within a range of 1μm and 4um; at times within a range of 2 μm and 4 μm. This size range was found to be particularly suitable for IM or SC injection.

In some further examples, the particles are defined by their d50 value (i.e. the dimensions of at least 50% of the particles) being within a range of 1 μm and 10 μm; at times within a range of 2 μm and 7 μm; at times within a range of 2.5 μm6 μm. In yet some further examples, the particles are defined by their d10 value (i.e. the dimensions of at least 10% of the particles) being within a range of 0.5 μm and 4 μm; at times within a range of 0.5 μm and 3 μm; at times within a range of 0.5 μm and 2μm.

An advantage of the presently disclosed formulation may reside in the dispersibility of the bound entity, being homogenously dispersed. The homogeneity of dispersion can be determined visually, e.g. where no significant amount of the cannabinoid powder is adhered to the surface of the vessel holding the dispersion and when observed under the microscope, the image exhibits homogenous particles with no particles that are related to CBD itself (the latter appearing under the microscope as large sticks or large crystals).

A further advantage of the presently disclosed formulation may reside in the particles size, being in the lower end of the micron range. Such small dimensions make the formulation particularly suitable for extravascular injection, as further discussed below.

The formulation can also be characterized by the absence of organic solvents, even trace amounts thereof. As appreciated, one hitherto used method for dissolving cannabinoids, involves the use of organic solvents, such as ethanol. The present disclosure allows for the formation of water based, high concentration, cannabinoid formulation without the use of organic solvents, and specifically without the use of ethanol. Thus, the formulations organic solvent free formulations.

In some examples, the formulation comprises specifically serum albumin, more specifically human serum albumin (HSA) and CBD.

In some further examples, the formulation comprises specifically serum albumin, more specifically non-human mammal albumin and CBD.

The protein-bound cannabinoid can be delivered in free form within the formulation, or as part of a delivery vehicle which is other than liposomes. For example, the protein-bound/associated cannabinoid can be embedded in a core-shell microcapsule structure, or in hydrogel of a type known in the art .

As noted above, the formulations are particularly suitable for injection, i.e. for use as an injectable formulation.

In the context of the present invention, when referring to an injectable formulation it is to be understood as encompassing any one of intramuscular (IM), subcutaneous (SC), intravenous (IV) injection, as well as infusion.

In one preferred example, the injectable formulation is suitable for IM injection.

In some other examples, the injectable formulation is suitable for SC injection. In some other examples, the injectable formulation is suitable for IV injection.

The formulation disclosed herein are obtainable by an intense/vigorous mixing process on the cannabinoid powder within an aqueous medium holding the protein, results in the effective dispersion of the cannabinoid within the aqueous medium.

Based on the above finding, a method has been developed, for obtaining protein-bound cannabinoid entities, with a weight ratio between the cannabinoid to protein being at least 10:50 (W/W) (e.g. 10 mg CBD to 50 mg HSA).

It is to be noted, however, that the present disclosure should not be limited to the method disclosed herein and in the context of the present disclosure the formulations are obtainable or obtained by the method disclosed herein.

In the context of the present disclosure, when referring to vigorous mixing stage it is to be understood as including at least one stage where the mixing involved at least one additional process parameter. In some examples, the process parameter includes duration of mixing that is effective to cause the formation of the association between the cannabinoid and protein (e.g. particulate/complex) as evident from simple imaging techniques. In some examples, the duration is at least several minutes, e.g. at least 30 minutes, at times at least 1 hour, at times, at least several hours (e.g. at least 5 hours).

In some examples the process parameter includes a mechanical parameter, such as velocity of mixing (rounds per minute) and/or homogenization. In some examples the process parameter includes a physical parameter, such as vibration, ultrasonic vibration.

In some examples, the at least one vigorous mixing stage comprises mixing under high shear or high pressure. In some examples, the high-shear or pressure comprises homogenization (Polytron, Kinematica AG PT2100 and SPX 1000/2000)

In some examples, the at least one vigorous mixing stage comprises applying ultrasonic vibration (e.g. sonication, using a bath or probe sonicator).

In some examples, the method comprises two or more distinguishable mixing processes, each defined by a different vigorous mixing stage.

In some examples, the method comprises at least one mixing step comprising ultrasonic irradiation and at least one mixing step comprising mixing for a time sufficient, under ultrasonic irradiation, to cause the association between the protein and the cannabinoid.

The formulation disclosed herein may be used for various applications.

In some preferred cases, the formulation is for use in a method of treatment. Thus, the present disclosure also provides a method of treatment comprising administering to a subject in need of the treatment the formulation disclosed herein.

In one example, the formulation is prolonged delivery of the cannabinoids, i.e. providing prolonged release provide when administered by injection.

In some examples, the method comprises administration of the formulation by injection.

Injection can include, in the context of the present disclosure, any form of injection, including IM, IV, SC. The route of administration can depend also on the size of the particles, where smaller particles (e.g. less than 300 nm) would typically be more suitable for the IV injection.

In some examples, the method comprises administration of the formulation by IM injection.

In some examples, the method comprises administration of the formulation by SC injection.

In some examples, the method comprises administration of the formulation by IV injection.

The non-limiting examples provided herein show that at least IM and SC injections exhibited a prolonged/extended duration release profile providing an advantage in the delivery of cannabinoids.

In some examples, the method comprises administration of the formulation to a mammalian subject.

In some examples, the method comprises administration of the formulation to a human subject.

In some other examples, the method comprises administration of the formulation to a non-human (i.e. veterinary) subject.

In view of the above, in the context of the present disclosure, when referring to treatment by the formulation disclosed herein, it is to be understood as encompassing ameliorating undesired symptoms associated with a disease, preventing the manifestation of such symptoms before they occur, slowing down the progression of a disease, slowing down the deterioration of symptoms, enhancing the onset of remission period of a disease, slowing down irreversible damage caused in progressive chronic stages of a disease, delaying onset of progressive stages, lessening severity or cure a disease, improving survival rate or more rapid recovery from a disease, preventing the disease from occurring, or a combination of two or more of the above.

The amount of the cannabinoid in the liposomes is designed to be sufficient to provide a therapeutic effect upon administration of the formulation to a subject.

An amount sufficient or effective to achieve a therapeutic effect upon administration is to be understood as including at least one therapeutic effect known to be achieved by or associated with cannabinoid compounds, particularly with CBD.

Without being limited thereto the therapeutic effect can be in any one or combination of treating/ameliorant/reducing pain and/or inflammation, as well as any other therapeutic effect known to be associated with the administration of cannabinoid compounds, particularly CBD.

The amount of cannabinoid compound to be delivered by the disclosed formulation depends on various parameters as known to those skilled in the art and can be determined based on appropriately designed clinical trials (dose range studies) and the person versed in the art will know how to properly conduct such trials in order to determine the effective amount. The amount depends, inter alia, on the type and severity of the disease to be treated and the treatment regime (mode of administration), gender and/or age and/or weight of the treated subject, etc.

As used herein, the forms “a”, “an” and “the” include singular as well as plural references unless the context clearly dictates otherwise. For example, the term “a cannabinoid” includes one or more cannabinoids.

Further, as used herein, the term “comprising” is intended to mean that the formulation comprises the protein and the cannabinoid, but not excluding other elements, such as physiologically acceptable carriers and excipients as well as other agents. The term “consisting essentially of” is used to define, for example, formulations which include the recited elements but exclude other elements that may have an essential significance on the delivery of CBD. “Consisting of” shall thus mean excluding more than trace elements of such other elements. Embodiments defined by each of these transition terms are within the scope of this invention.

Further, all numerical values, e.g. when referring the amounts or ranges of the elements constituting the liposomes and formulations comprising the same are approximations which are varied (+) or (−) by up to 20%, at times by up to 10% of from the stated values. It is to be understood, even if not always explicitly stated that all numerical designations are preceded by the term “about”.

The invention will now be described by way of non-limiting examples that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### DETAILED DESCRIPTION OF NON-LIMITING EXAMPLES

### Example 1—Protein Bound CBD

### Materials

The materials used for the formulations are found in Table 1.

TABLE 1
Materials

| Material | Details |
|---|---|
| CBD | THC Pharma, Batch CBDAPI1805 |
| Human Serum Albumin (HSA) | Alburex 50 g/l, lot P100020267 |
| Immunoglobulins, IVIg | Grifols, Flebogamma 5% DIF, lot |
| | A4GDD00541, Exp Aug. 2021 |

### Formulation preparations

Three methods were tested for achieving homogenous dispersions of CBD 50 mg/ml in 5% HSA (50 mg/ml):

1) stirring for ≥48 h (maximum speed of laboratory stirrer), 2) ultrasonic irradiation in a bath sonicator (S 30 H Elmasonic) at room temperature (RT, 25° C.±2° C.), for 1 minute (unless otherwise stated), and

3) Homogenization (Polytron, Kinematica AG PT2100).

In the following non-limiting examples, when referring to the concentration of CBD in the protein bound CBD structures, e.g. CBD-HSA, only the weight of CBD is defined and should be understood to refer to the weight of CBD for 50 mg/ml of the recited protein. Thus, CBD 50mg/ml should be understood to refer to 50 mg CBD and 50 mg HSA per ml. Similarly, CBD-HSA (100 mg/ml) is to be understood as referring to 100 mg CBD and 50 mg HSA.

CBD-HSA preparations following 1 h of stirring showed many large particles (FIGS. 1A-1E). These large particles disappeared after further stirring for up to 24 h with additional improvement towards more homogenous and smaller particles after 48 h of stirring. Stirring for 10 days showed further reduction in size.

The formulations were observed under optical microscope (Zeiss SN 221209). Few fields were observed, and a representative picture was taken for each formulation.

Specifically, FIGS. 1A-1C, which are microscope images (×200 magnification) show the level of homogeneity of CBD-HSA 50 mg/ml formulations after 1 hour of stirring at room temperature (25° C.), 24 hours of stirring at 4° C. and 48 hours stirring at 4° C., respectively. FIG. 1D shows CBD-HSA (100 mgCBD/50mgHSA) after stirring at 4° C., for 48 hours. FIG. 1E shows CBD-HSA (250mgCBD/50 mgHSA) after stirring at 4° C., for 10 days.

Another method to achieve particle homogeneity included homogenization.

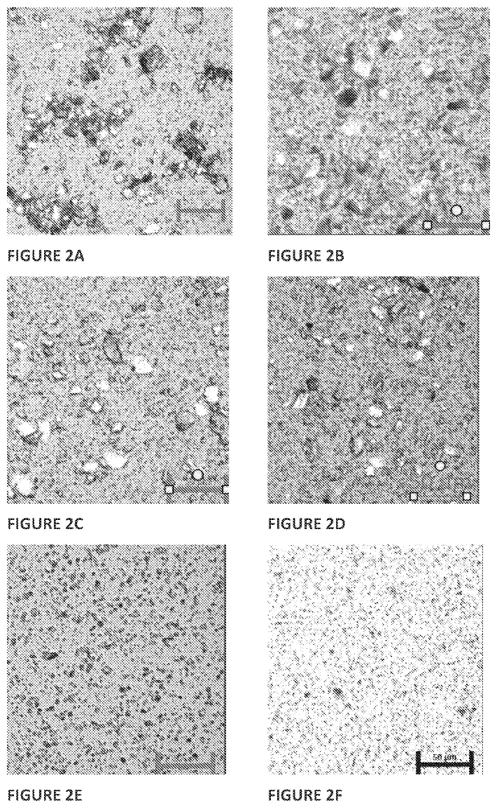

Specifically, 50 mg/g CBD-HSA formulation (1 ml) was subjected to intense homogenization under conditions equivalent to 11,000-18,000 rpm (homogenizer: Polytron, Kinematica AG PT2100). Homogenization was carried out in an ice bath and images were taken after different time points (5 minutes, 10 minutes, 15 minutes, 20 minutes, 30 minutes and 60 minutes). The results are presented in FIGS. 2A-2F. Specifically, FIG. 2A shows CBD-HSA (50 mg/g) following homogenization, taken after different durations, including 5 minutes, FIG. 2B shows the result of homogenization after 10 minutes, FIG. 2C shows the result of homogenization after 15 minutes, FIG. 2D shows the result of homogenization after 20 minutes, FIG. 2E shows the result of homogenization after 30 minutes and FIG. 2F shows the result of homogenization after 60 minutes.

As shown in FIGS. 2A-2F, the appearance of the formulation by the microscope after 30 minutes or 60 minutes homogenization was similar to that obtained after 48 hours stirring. Particle size (as measured by Coulter LS 130) was 3.1 μm and 2.7 μm, respectively. Thus, it is evident that the duration of applying the conditions for forming the association/complex will depend on the selected condition parameter, be it, for example, vigorous mixing, homogenization etc.

Yet, another method for achieving homogenous dispersion, included ultrasonic irradiation using a bath sonicator at RT.

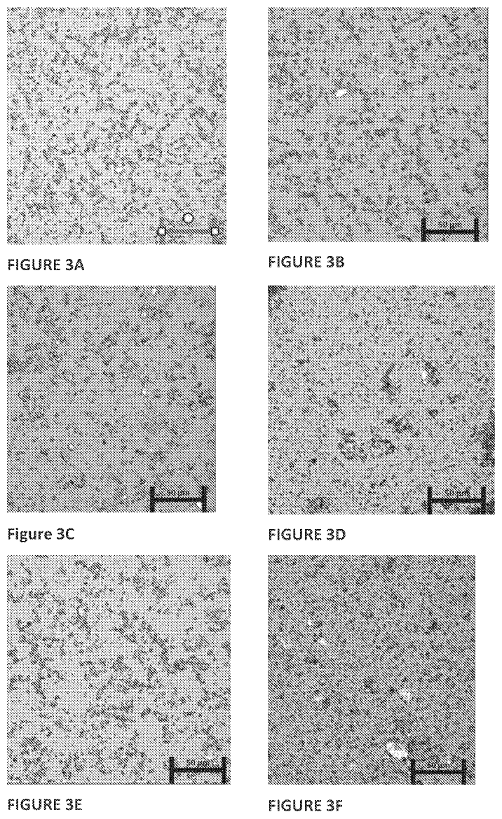

The results are presented in FIGS. 3A-3F. Specifically, FIG. 3A shows CBD-HSA (50 mg/ml) following 1 minute of sonication, FIG. 3B shows the result after minutes of sonication, FIG. 3C shows the result after 15 minutes of sonication, FIG. 3D shows the result after 20 minutes of ultrasonic irradiation, FIG. 3E shows the result after 30 minutes of ultrasonic irradiation and FIG. 3F shows the result after minutes of ultrasonic irradiation

Relatively homogeneous particle population of about Sum mean diameter were obtained even after 1 minute of ultrasonic irradiation. Interestingly, increasing sonication time did not show significant improvement.

### Maximal CBD Loading and Particle Size

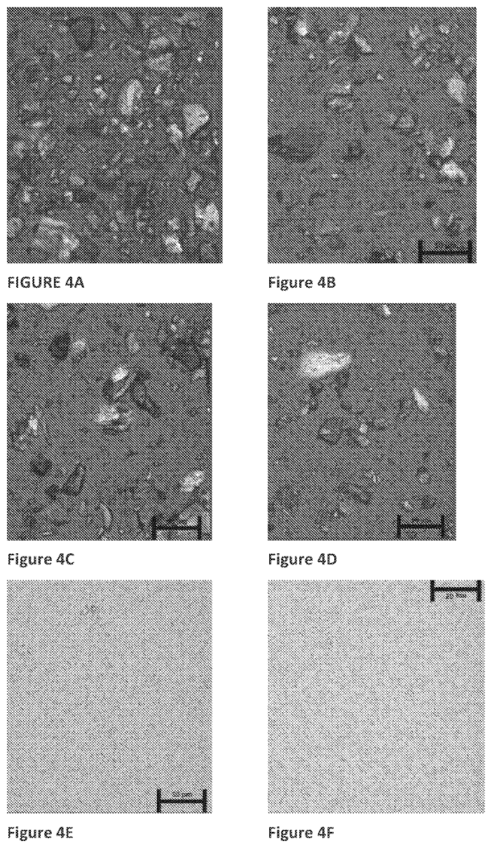

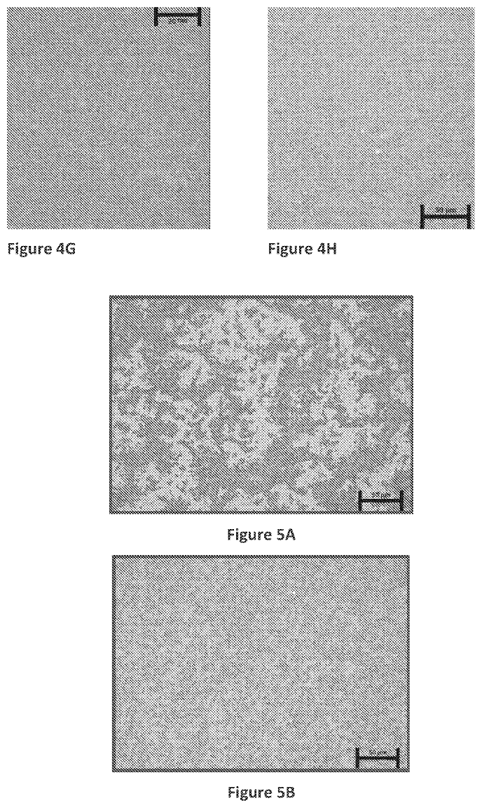

CBD at concentrations of 100-400 mg/ml in 5% HSA solution (HSA at a constant concentration of 50 mg/ml) were prepared. The preparations were exposed to ultrasonic irradiation for 1 min (and a sample was taken) followed by 48 h of stirring at 4≥C which further reduced particle size as described in Table 2. The appearance of the preparations after 1 min of ultrasonic irradiation and after additional 48 h of stirring at 4 C (×200 magnification) are described in FIGS. 4A-4H.

Mean particle size determined by Coulter LS130 are found in Table 2. The results show that high concentrations of 100-400 mg/ml preparations resulted, after 1 min of ultrasonic irradiation and 48 h of stirring, achieving homogenous suspensions having mean particle size in the range of 2.2-2.9 μm.

TABLE 2
Particle size (μm) after different preparation methods

| CBD amount* | 100 mg/ml | 200 mg/ml | 300 mg/ml | 400 mg/ml |
|---|---|---|---|---|
| After 1 min ultrasonic | 17.92 | 14.84 | 15.71 | 11.33 |
| irradiation | | | | |
| After 1 min ultrasonic | 2.34 | 2.16 | 2.86 | 2.53 |
| irradiation and 48 h | | | | |
| stirring | | | | |

*for 50 mg protein

### CBD-HSA Lyophilization

A formulation of HSA-CBD (167 mg/g) was lyophilized Lyophilization cycle is described herein: Vials were loaded onto a shelf of the sublimation chamber at 18° C. The shelf was cooled to −50° C. The preparation was cooled from ˜36 to —40° C. and held at that temperature for 3 h (freezing stage). The shelf was held at minus 50° C. while the pressure in the chamber was reduced (4.0-6.0)×10⁻² mBar for 2 h and heated from −50 to +20° C. at 6° C./ h (sublimation drying). The preparation was warmed to 20° C. (pressure in chamber 4.0×10 ⁻² mBar) and held at that temperature for 2 h (drying). The total drying time was 19 h.

After lyophilization, the sample was rapidly hydrated with Double distilled water (DDW) and the appearance of the samples by the microscope before and after lyophilization is presented in FIG. 5A and FIG. 5B, respectively. Size and free vs bound CBD before and after lyophilization are provided in Tables 3A and 3B, respectively.

TABLE 3A
Particle size before and after lyophilization

| | Size (μm) | | | | | | | |
|---|---|---|---|---|---|---|---|---|
| Lyophilization | Mean | Median | SD | <10% | <25% | <50% | <75% | <90% |
| Before | 4.580 | 2.946 | 4.031 | 0.824 | 1.346 | 2.946 | 6.981 | 11.34 |
| After | 5.015 | 3.357 | 4.341 | 0.886 | 1.497 | 3.357 | 7.652 | 12.11 |

TABLE 3B
Release in serum of formulation before and after lyophilization

| | % Free | % Bound |
|---|---|---|
| Before lyophilization | 10.32 | 89.68 |
| After lyophilization | 9.57 | 90.43 |

It was thus concluded that the formulations are suitable for lyophilization.

In conclusion, CBD-HSA complex was found to be suitable for lyophilization.

### Size Reduction

CBD-HSA preparations (333 and 429 mg/g) were stirred for 23 days. Size by

Coulter is summarized in Table 4.

TABLE 4
Size of HSA-CBD formulations stirred for 23 days

| | Size (μm) | | | | | | | |
|---|---|---|---|---|---|---|---|---|
| | Mean | Median | SD | <10% | <25% | <50% | <75% | <90% |
| HSA-CBD | 0.739 | 0.731 | 0.171 | 0.515 | 0.608 | 0.731 | 0.862 | 0.977 |
| 333 mg/g | | | | | | | | |
| HSA-CBD | 0.86 | 0.852 | 0.211 | 0.583 | 0.701 | 0.852 | 1.013 | 1.147 |
| 429 mg/g | | | | | | | | |

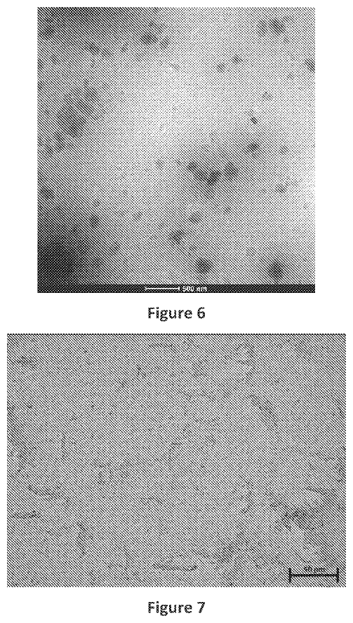

The small CBD-HSA particles were observed by CryoTEM as provided in FIG. 6. The above results show that it is possible to reduce the particles size without damaging the protein-CBD assembly. After repeated centrifugations it was possible to isolate particles of 130-140 nm diameter. These small particles may be used for IV injection.

Canine albumin (CA)-CBD particles

CA-CBD (50 mg/g) was prepared as described with HSA, and stirred at 4° C. Size was measured at several time points (D=Days, W=Weeks) as described in Table 5.

TABLE 5
CA-CBD size over stirring time

| | Size (μm) | | | | | | | |
|---|---|---|---|---|---|---|---|---|
| Time | Mean | Median | SD | <10% | <25% | <50% | <75% | <90% |
| 5 D | 2.886 | 2.366 | 1.853 | 0.971 | 1.453 | 2.366 | 3.922 | 5.672 |
| 6 D | 2.583 | 2.085 | 1.668 | 0.934 | 1.344 | 2.085 | 3.398 | 5.078 |
| 8 D | 2.216 | 1.82 | 1.38 | 0.875 | 1.225 | 1.82 | 2.804 | 4.221 |
| 2 W | 1.898 | 1.606 | 1.131 | 0.824 | 1.126 | 1.606 | 2.315 | 3.361 |

Table 5 shows that it is possible to obtain particles of similar properties, including size and shape when using albumin from a different species (not limited to human).

### Mouse Albumin (MA)-CBD Particles

Similarly, mouse albumin CBD particles (MA-CBD, 50 mg/g) were prepared and stirred at 4° C.

Particles' size was measured following 5 days of stirring, and the result is summarized in Table 6.

TABLE 6
MA-CBD size over stirring time

| | Size (μm) | | | | | | | |
|---|---|---|---|---|---|---|---|---|
| | Mean | Median | SD | <10% | <25% | <50% | <75% | <90% |
| 5 days | 6.208 | 5.186 | 4.573 | 1.006 | 2.163 | 5.186 | 9.585 | 13.27 |

Table 6 shows that it is possible to obtain particles of similar properties, including size and shape when using albumin from a different species (not limited to human). The appearance of the formulation after 5 days of stirring is provided in FIG. 7.

### CBD-IVIg

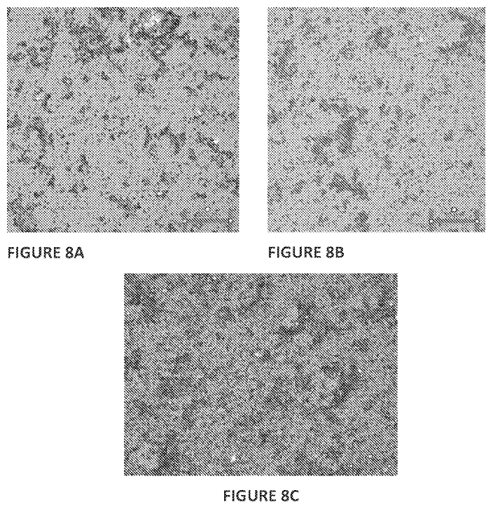

In a similar manner, homogenous preparations of CBD in 5% immunoglobulin solution (IVIg) were prepared upon 24 or 48 h of stirring at 4° C. FIG. 8A-8C are images of CBD-IVIg (50mg/m1) after 24 hours of stirring at 4° C. and 48 hours stirring at 4° C., respectively.

Preparation of CBD complexes with IVIg shows that other proteins are suitable for obtaining complexes with CBD.

### Release of CBD from CBD-HSA Formulations

The release of CBD from the different formulations was tested in 50% adult bovine serum. In an HPLC vial, 50 mg formulation was weighed and 950 ul of 50:50 of serum: dextrose 5% solution was added

The mixture was vortexed and placed in an incubator at 37° C. and 50 rpm shaking for 2 hr. The mixture was tested for total CBD content after dilution of 25-fold in methanol. The rest of the mixture was transferred to an Eppendorf and centrifuged (30 min, 14,000 rpm, 4° C.) and the upper phase was diluted 10-fold in methanol and HPLC analyzed. Table 7 presents the release from different CBD formulations. The results showed that the maximal free CBD released after incubation with 50% serum was similar for all preparations and ranged between 14.1-15.3 mg/ml corresponding to 14-33% release. It is noted that the free CBD in the formulation was very low <0.3 mg/ml.

TABLE 7
CBD % release

| | | Free CBD conc. |
|---|---|---|
| Formulation | % Released | (mg/ml) |
| CBD-HSA 50 mg/ml | 28 | 14.84 |
| CBD-HSA 100 mg/ml | 14 | 15.34 |
| CBDIVIg 50 mg/ml | 28 | 14.12 |
| CBD-IVIg 100 mg/ml | 14 | 14.70 |
| CBD-HSA 50 mg/ml | 33 | 14.25 |
| (30 min homogenization) | | |
| CBD-HSA 50 mg/ml | 31 | 14.79 |
| (60 min homogenization) | | |

### Example 2—In Vivo Study of CBD-HSA Formulations

### Formulations Preparation for In Vivo Study and Characterization

CBD-HSA 50 and 100 mg/ml and CBD:IVIg 50 and 100 mg/ml were prepared by weighing CBD into a vial, adding HSA or IVIg solution and stirring for 48 h at 4° C.

IV formulation: The formulation used for IV administration was 10 mg/g CBD formulation solubilized in Cremophor:Ethanol 50:50 solution. This formulation was diluted 10-fold with saline prior to injection to result in 1 mg/ml post dilution concentration. The diluted formulation was used within 1 h after preparation.

### Formulation Characterization

### Assay for CBD

Total and free CBD content was determined by HPLC method. The chromatographic conditions used were based on USP method for Dronabinol and summarized in Table 8.

Sample preparation for the analysis varied for each formulation and described below.

Sample preparation for the analysis varied for each formulation and described

Total CBD concentration was similar for all formulations. Specifically, 10-20 mg of formulation was weighed into a 10 ml volumetric flask. Methanol was added to line. After vortex, sample was centrifuged, and the upper phase was analyzed.

Free CBD content: A 200 μl formulation was placed in an Eppendorf and centrifuged for 30 min at 40° C., 14,000 rpm. The clear upper phase was then diluted 10-fold with methanol followed by vortex and centrifugation (14,000 rpm, 10 min, 40° C.). Upper phase was HPLC analyzed.

The IV formulation in Cremophore: ethanol was tested for total content as described above for Total CBD concentration. The appearance after dilution with saline was examined to follow formulation behavior for injection and ensure no precipitation. The formulation was diluted ×10 with saline and after 1 h (the time allowed for the formulation to be injected after preparation), the appearance was recorded.

TABLE 8
Chromatographic conditions of CBD assay

| | Methanol:DDW:tetrahydrofuran:acetonitrile |
|---|---|
| Mobile phase | at a volumetric ratio of 45:25:20:10 |
| Column | Phenomemex C18, 150 × 4.6 mm |
| Detector | UV 228 nm |
| Flow rate | 1 ml/min |
| Injection vol. | 20 μl |
| Column temp | 25° C. |

### Release Assay

The release of CBD from the different formulations was tested in 50% adult bovine serum. In an HPLC vial, 50 mg formulation was weighed and 950 ul of 50:50 of serum: dextrose 5% solution was added. The mixture was vortexed and placed in an incubator at 37 C and 50 rpm shaking for 2 hr. The mixture was tested for total CBD content after dilution of 25-fold in methanol. The rest of the mixture was transferred to an Eppendorf and centrifuged (30 min, 14,000 rpm, 4° C.) and the upper phase was diluted in methanol and HPLC analyzed.

### Particle Size Measurement

Particle size was determined using Coulter LS 130.

### Osmolality

Osmolality was measured by freeze point method using Advanced instrument, Model 3320 osmometer.

### Microscopical Observation

The formulations were observed under optical microscope (Zeiss SN 221209). Few fields were observed, and a representative picture was taken for each formulation.

### Injectability

One ml syringe was filled with 0.3-0.5 ml of formulation. A 25G needle was connected to the syringe and the injected volume of the formulation without being stuck was determined. The process was repeated for three times.

### Sterility

One vial from each formulation was tested by the microbiology unit in Hadassah. Aliquot from each vial was plated on blood agar and chocolate agar and placed at room temperature and at 37° C. incubator.

### In Vivo Study Protocol

### IV Administration

A total of 18 female BALB/C mice aged 12 weeks were injected IV with a single dose of 12 mg/kg CBD formulation in cremophor:ethanol.

At the time-points detailed below, 3 mice were euthanized with CO₂ and terminal blood was immediately collected from the retro-orbital sinus in labeled 0.5 ml K₃ EDTA blood collection tubes (Mini Collect, Greiner-bio-one, Austria). The blood was centrifuged at 2000×g for 10 minutes before plasma was extracted, collected in labeled tubes and frozen at −20° C. immediately after collection. The samples were then stored at −80° C. pending analysis.

Time-points for the blood collection: 2 min, 1, 4, 8, 24 and 48 h.

### IM Administration

A total of 37 female BALB/C mice aged 12 weeks were injected IM with a single dose of the CBD-HSA 50 and 100 mg/ml and CBD:IVIg 50 and 100 mg/ml as described in Table 9. Nine mice per formulation. The syringe was weighed before and after injection to enable precise recording of the exact volume and hence dose that each mouse received. Details regarding the injection volumes and estimated doses for each group are summarized in Table 9.

At the time-points detailed below, 3 mice of each group were euthanized with CO₂ and terminal blood sample was immediately collected from the retro-orbital sinus in labeled 0.5 ml K₃EDTA blood collection tubes (Mini Collect, Greiner-bio-one, Austria). The blood was centrifuged at 2000×g for 10 minutes before plasma was extracted, collected in labeled tubes and frozen at −20° C. immediately after collection. The samples were then stored at −80° C. pending analysis.

After blood collection, the site of administration which is quadriceps femoris muscles were collected into pre-weighed 15 ml tubes.

Time-points for the blood collection: 72 hours, 1 week and 3 weeks after injection.

Mice weight was recorded before dosing and before euthanization. Mice sacrificed at 3-weekd time point were also weighed two weeks after administration.

TABLE 9
Injection volumes and estimated doses for each group

| | Injection | No. of | Estimated dose |
|---|---|---|---|
| | volume per | injected | (mg/kg) assuming |
| Group | muscle (ml) | muscles | 20 g mouse |
| CBD-HSA 50 mg/ml | 0.05 | 1 | 145 |
| CBD-HSA 100 mg/ml | 0.05 | 1 | 293 |
| CBD-IVIg 50 mg/ml | 0.05 | 1 | 137 |
| CBD-IVIg 100 mg/ml | 0.05 | 1 | 275 |

### Bioanalytical Assays

### Assay for CBD in Plasma

CBD was extracted from plasma samples that were spiked with cannabigerol (CBG, 1 mg/ml in methanol, Sigma, Cat. C-141-1 Add lot) used as internal standard (IS) followed by dilution of the plasma five-fold in acetonitrile. After vigorous vortex it was centrifuged, and the upper phase was analyzed. Final IS concentration in the samples was 100 ng/ml.

Plasma extracts were analyzed by LC/MS method (Sciex (Framingham, MA, USA) Triple Quad™ 5500 mass spectrometer coupled with a Shimadzu (Kyoto, Japan) UHPLC System). The concentrations were calculated based on a calibration curve of CBD in plasma at a range of 1-1,000 ng/ml having 100 ng/ml of Internal Standard (IS).

CBD spiking solutions for the preparation of calibration curve in plasma were prepared in acetonitrile. CBG was dissolved in methanol.

### Assay for CBD in Muscles (Site of Injection)

Muscles were removed surgically, and their weight was recorded. Thereafter, 2 ml of 15% collagenase solution (Sigma, C7657) were added to degrade the tissue and tubes were incubated overnight at 37° C. After incubation, 8 ml of acetonitrile were added, vortexed and centrifuged. Upper phase was HPLC analyzed. Chromatographic conditions were described in Table 8.

The concentration of CBD in each muscle was calculated based on a calibration curve of CBD in acetonitrile.

The recovery of CBD from muscles was determined for each formulation following spiking of CBD formulations into muscles compared to spiking into acetonitrile.

### Results

### Formulations

FIGS. 1A-1B and FIGS. 8A-8C provide microscopic images of the suspensions, and shows that the obtained particles are small, and that the appearance of the HSA based particles (FIG. 1A-1D) was different from the IVIg particles (FIG. 8A-8C).

The formulations characterization is provided in Table 10 and the particle size is summarized in Table 11.

The microscope observation was also supported by the size, as detailed in Table 7 below.

The release of CBD in the presence of serum was similar for these two formulations, having 14-15 mg/ml free CBD after 2 hr of incubation in 50% serum accounting for 28% release for the 50 mg/g formulations and 14% release for the 100 mg/g formulations

TABLE 10
Formulation characterization (for IM administration)

| | | | Release | |
|---|---|---|---|---|
| | Total CBD | Free CBD* | assay:% | |
| | (mg/ml) | (mg/ml) | Bound** | Injectability |
| CBD-HSA 50 mg/ml | 58.01 | 0.135 | 72 | very good |
| CBD-HSA 100 mg/ml | 117.17 | 0.146 | 86 | very good |
| CBD-IVIg 50 mg/ml | 54.64 | 0.032 | 72 | very good |
| CBD-IVIg 100 mg/ml | 109.96 | 0.261 | 86 | very good |

*Free CBD concentration in the formulation
**Bound after 2 hr, in 50% serum. Release assay conditions

It is noted that all formulations were also found to be sterile, i.e. no microbial growth was detected in any of the tested formulations.

The CBD formulation in Cremophor:ethanol for IV administration was also characterized. The concentration of CBD in the concentrate was 11.7 (mg/ml). After dilution with saline, the solution was clear for at least 1 hour.

TABLE 11
Particle size

| Formulation | Mean | <10% | <50% | <90% |
|---|---|---|---|---|
| CBD-HSA 50 mg/ml | 7.0 | 1.1 | 5.5 | 15.5 |
| CBD-HSA 100 mg/ml | 5.2 | 1.0 | 3.6 | 12.6 |
| CBD-IVIg 50 mg/ml | 4.5 | 0.9 | 3.1 | 10.6 |
| CBD-IVIg 100 mg/ml | 3.8 | 0.9 | 2.6 | 9.0 |

The PK profile obtained after IV administration of 12 mg/kg CBD dose is summarized in Table 12.

TABLE 12
Plasma CBD profile after IV administration
of 12 mg/kg dose of free CBD

| | |
|---|---|
| Time point | Mean ± SD |

| | | |
|---|---|---|
| 5 | min | 8,856 ± 1,451 |
| 1 | h | 177 ± 40 |
| 4 | h | 35 ± 11 |
| 8 | h | 9.50 ± 3.67 |
| 24 | h | BLOD |
| 48 | h | BLOD |

*BLOD—below limit of detection

Table 12 shows that CBD concentrations decreased rapidly from 8,856 ng/ml at 5 min to 9.5 ng/ml, 8 hr after administration. At the late time points (24 hr and 48hr) CBD concentrations were below the limit of detection (BLOD).

Plasma concentrations obtained after IM administration are summarized in Table 13 and FIG. 9.

TABLE 13
CBD plasma concentrations across IM formulations

| | | | | Plasma conc. | Mean (±SD) |
|---|---|---|---|---|---|
| | Time | CBD | Plasma | Normalized | plasma conc. |
| | point | dose | conc. | to dose | Normalized to dose |
| Formulation | (days) | (mg/kg) | (ng/ml) | (ng/ml/mg/kg)* | (ng/ml/mg/kg)** |
| CBD-HSA | 3 | 163 | 27 | 0.167 | 0.185 ± 0.042 |
| 50 mg/ml | | 151 | 35 | 0.234 | |
| | | 156 | 24 | 0.155 | |
| | 7 | 136 | 26 | 0.190 | 0.138 ± 0.045 |
| | | 163 | 19 | 0.120 | |
| | | 158 | 17 | 0.105 | |
| | 21 | 173 | 9.0 | 0.052 | 0.065 ± 0.032 |
| | | 170 | 17 | 0.102 | |
| | | 161 | 6.8 | 0.042 | |
| CBD-HSA | 3 | 317 | 41 | 0.129 | 0.166 ± 0.038 |
| 100 mg/ml | | 327 | 54 | 0.165 | |
| | | 343 | 70 | 0.205 | |
| | 7 | 336 | 22 | 0.066 | 0.082 ± 0.017 |
| | | 433 | 34 | 0.079 | |
| | | 327 | 32 | 0.099 | |
| | 21 | 350 | 5.8 | 0.017 | 0.022 ± 0.006 |
| | | 334 | 6.8 | 0.020 | |
| | | 303 | 8.4 | 0.028 | |
| CBD-IVIg | 3 | 145 | 9.2 | 0.064 | 0.129 ± 0.062 |
| 50 mg/ml | | 184 | 25 | 0.135 | |
| | | 159 | 30 | 0.187 | |
| | 7 | 150 | 24 | 0.163 | 0.138 ± 0.050 |
| | | 153 | 12 | 0.080 | |
| | | 159 | 27 | 0.170 | |
| | 21 | 141 | 5.3 | 0.038 | 0.064 ± 0.024 |
| | | 134 | 11 | 0.085 | |
| | | 197 | 14 | 0.071 | |
| CBD-IVIg | 3 | 310 | 43 | 0.140 | 0.187 ± 0.067 |
| 100 mg/ml | | 271 | 63 | 0.234 | |
| | 7 | 370 | 33 | 0.089 | 0.092 ± 0.025 |
| | | 319 | 38 | 0.118 | |
| | | 320 | 22 | 0.068 | |
| | 21 | 291 | 13 | 0.044 | 0.053 ± 0.014 |
| | | 309 | 21 | 0.069 | |
| | | 268 | 12 | 0.046 | |

*Plasma concentration normalized to dose (ng/ml/mg/kg)
**Mean ± SD plasma concentration normalized to dose (ng/ml/mg/kg)

Table 13 and FIG. 9 show that the plasma concentrations after administration of all IM formulations and up to 3 weeks after administration were in the range of the IV profile obtained 1-8 hr after administration. This means, that these protein-CBD formulations, when injected IM, exhibit a prolongation of CBD blood concentration at a level that based on CBD level after daily IV injection. Therefore, such IM administration of these formulations should enable prolongation of the therapeutic effects.

Moreover, the reduction in plasma CBD levels was very slow with less than one order of magnitude decrease obtained over 3 weeks, for all formulations. This slow decrease, compared to the rapid decrease of the IV formulation, demonstrates that the terminal slope of the IM profile is not elimination dependent but rather absorption dependent showing that the formulations are continuously releasing CBD from the muscles over this long period.

The best proof to it is that as shown in Table 14 below after 3 weeks still significant portion of the CBD remain at the site of administration. Namely, it served as a CBD reservoir.

Table 14 summarizes residual content of CBD in the muscles compared to the initial CBD administered to each mouse. FIGS. 10A-10B presents, respectively, the absolute CBD release (mg) and % CBD released, from the muscles per group compared to the initial administered CBD.

TABLE 14
CBD muscle concentrations (N = 3 per group)

| | | | | | CBD | | Plasma CBD |
|---|---|---|---|---|---|---|---|
| | | | | | released | | normalized to |
| | | Absolute | Absolute | % CBD | from | Estimated | estimated |
| | | CBD | CBD in | released | muscle | daily | CBD daily |
| | Time | administered | muscle | from | per day | CBD dose | dose (ng/ml/ |
| Formulation | (days) | (mg) | (mg) | muscle^{a} | (mg/day) | (mg/kg)^{b} | mg/kg) |
| CBD-HSA | 7 | 2.69 | 1.88 | 30 | 0.12 | 6.0 | 4.33 |
| 50 mg/ml | | 3.37 | 2.46 | 27 | 0.13 | 6.5 | 2.92 |
| | | 3.14 | 2.20 | 30 | 0.13 | 6.5 | 2.62 |
| | 21 | 3.06 | 1.84 | 40 | 0.06 | 3.0 | 3.00 |
| | | 3.14 | 2.33 | 26 | 0.04 | 2.0 | 8.50 |
| | | 3.16 | 1.26 | 60 | 0.09 | 4.5 | 1.51 |
| CBD-HSA | 7 | 6.32 | 4.16 | 34 | 0.31 | 15.5 | 1.42 |
| 100 mg/ml | | 7.82 | 4.88 | 38 | 0.42 | 21.0 | 1.62 |
| | | 6.48 | 4.57 | 29 | 0.27 | 13.5 | 2.37 |
| | 21 | 6.55 | 3.43 | 48 | 0.15 | 7.5 | 0.77 |
| | | 5.83 | 5.25 | 10 | 0.03 | 1.5 | 4.53 |
| | | 5.69 | 4.63 | 19 | 0.05 | 2.5 | 3.36 |
| -CBD-IVIg | 7 | 2.99 | 1.86 | 38 | 0.16 | 8.0 | 3.00 |
| 50 mg/ml | | 3.07 | 2.43 | 21 | 0.09 | 4.5 | 2.67 |
| | | 3.09 | 2.18 | 29 | 0.13 | 6.5 | 4.15 |
| | 21 | 2.88 | 2.13 | 26 | 0.04 | 2.0 | 2.65 |
| | | 2.63 | 1.48 | 44 | 0.05 | 2.5 | 4.40 |
| | | 3.25 | 1.45 | 55 | 0.09 | 4.5 | 3.11 |
| CBD-IVIg | 7 | 7.13 | 5.87 | 18 | 0.18 | 9.0 | 3.67 |
| 100 mg/ml | | 6.07 | 5.16 | 15 | 0.13 | 6.5 | 5.85 |
| | | 6.20 | 6.22* | 0 | 0 | 0.0 | NC |
| | 21 | 5.89 | 3.01 | 49 | 0.14 | 7.0 | 1.86 |
| | | 6.03 | 2.79 | 54 | 0.15 | 7.5 | 2.80 |
| | | 5.27 | 4.40 | 16 | 0.04 | 2.0 | 6.00 |

${}^{a} \% CBD released from muscle = \frac{CBD administered - CBD in muscle}{CBD administered} \times 100$ ^{b}Estimated daily CBD dose (mg/kg) calculated for 20 g mouse = CBD released per day/0.02 kg

At 1-week time point, differences were found between groups showing more release for the high dose groups. These differences were not found for the 3-weeks' time point.

From the % CBD release data, CBD reservoir in the muscle may be calculated for each formulation. The HSA and IVIg formulations did not release half of the CBD content in the muscles over 3 weeks. This provides further evidence to the prolonged release of CBD by the disclosed formulations.

### Example 3—In Vivo PK Study of CBD-Mouse and Canine Albumin Formulations

### Mice Albumin (MA) CBD Formulation

Mice were injected SC with either CBD-HSA or CBD-MA at two different dosages, 125 mg/kg and 250 mg/kg. The plasma profile obtained is presented in FIG. 11 (it is noted that plasma profile data of CBD-HSA at 125 mg/kg at the first time points, 2-72 h, is taken from a previous study).

The results show that there is no difference between the prolonged release obtained when using albumin from human (HSA) or mice, upon injection to mice.

### Canine-Albumin (CA) CBD Formulation

Russian terrier dog aged 10 years suffering from Melanoma on HL paw was injected with CBD-CA at a dose of 5 mg/kg (dog weight on injection day—55 kg). Four days after the injection the dog was euthanized because the melanoma ruptured, bled and the dog was painful.

Plasma samples were taken at several time points following injection and the profile is provided in FIG. 12. The results show prolonged release of CBD even after 4 days.

The in vivo PK data of mice injected with CBD-MA and dog injected with CBD-CA, demonstrated that using CBD-albumin formulations that are compatible with the injected animal, results in the similar prolonged release profile obtained previously when CBD-HSA was injected to mice.

1. A formulation comprising a holding medium and homogeneously dispersed therein protein-bound cannabinoid, wherein said cannabinoid and said protein in the protein-bound cannabinoid are at a weight ratio of at least 10 mg cannabinoid to 50 mg protein (10:50).

2. The formulation of claim 1, wherein said cannabinoid and said protein are present in the protein-bound cannabinoid at a weight ratio within a range of 30:50 and 500:50.

3. The formulation of claim 1, wherein said protein-bound cannabinoid is in a form of particles having a mean particle size of up to 10 μm.

4. (canceled)

5. The formulation of claim 1, wherein said protein-bound cannabinoid is in a form of particles with at least 90% of the particles having a particle size of less than 20 μm.

6. The formulation of claim 1, wherein said protein is a serum derived protein.

7. The formulation of claim 1, wherein said protein is selected from albumin and globulins.

8. The formulation of claim 7, wherein said protein is albumin.

9-10. (canceled)

11. The formulation of claim 7, wherein said protein is selected from alpha globulins, beta globulins and gamma globulins.

12. (canceled)

13. The formulation of claim 1, wherein said cannabinoid comprises cannabidiol (CBD) or a functional homologue thereof.

14-15. (canceled)

16. The formulation of claim 1, being free of unbound cannabinoid.

17-20. (canceled)

21. A method of obtaining protein-bound cannabinoid particles, the method comprises mixing an aqueous medium of said protein with at least cannabinoid compound to form a mixture, wherein said mixing comprises at least one vigorous mixing stage at a cannabinoid to protein weight ratio of at least 10:50.

22. The method of claim 21, wherein said at least one vigorous mixing stage comprises mixing of the mixture for a time sufficient to cause non-covalent association between said protein and said cannabinoid.

23. The method of claim 21, wherein said at least one vigorous mixing stage comprises homogenization.

24. The method of claim 22, wherein said at least one vigorous mixing stage comprises applying ultrasonic vibration.

25. The method of claim 21, wherein said mixing is in the absence of an organic solvent.

26. The method of claim 21, wherein said mixing is in the absence of ethanol.

27. A method of treatment, the method comprising administering to a subject in need of said treatment a formulation according to claim 1.

28. The method of claim 27, wherein said administration comprises injection of said formulation.

29. The method of claim 28, wherein said injection comprises intramuscular (IM) injection.

30. The method of claim 28, wherein said injection comprises subcutaneous (SC) injection.
